(19) United States
(12) Patent Application Publication
(10) Pub. No.: US 2023/0242539 A1
(43) Pub. Date: Aug. 3, 2023

(54) Polymorphs of (1S,2S,3S,5R)-3-((6-(Difluoromethyl)-5-Fluoro-1,2,3,4-Tetrahydroisoquinolin-8-YL)OXY)-5-(4-Methyl-7H-Pyrrolo[2,3-D]Pyrimidin-7-YL)Cyclopentane-1,2-DIOL Mono-Hydrochloride

(71) Applicant: Pfizer Inc., New York, NY (US)

(72) Inventors: Michael Heberlein, Poway, CA (US); Klimentina Dimitrova Pencheva, Sandwich Kent (GB); Michelle Bich Tran-Dubé, San Diego, CA (US)

(73) Assignee: Pfizer Inc., New York, NY (US)

(21) Appl. No.: 18/004,965

(22) PCT Filed: Jul. 8, 2021
(86) PCT No.: PCT/IB2021/056157

§ 371 Date: Jan. 10, 2023

Related U.S. Application Data
(60) Provisional application No. 63/051,977, filed on Jul. 15, 2020.

(51) Int. Cl. C07D 487/04

(52) U.S. Cl. CPC C07D 487/04; C07B 2200/13

(57) ABSTRACT

This invention relates to crystalline mono-HCl salt forms of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol, and to compositions and therapeutic uses thereof.

### FIELD OF THE INVENTION

The present invention relates to a novel crystalline mono-HCl salt form (“Form 4”) of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol. The present invention also relates to formulations and therapeutic uses of such polymorph.

### BACKGROUND

The compound (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7-yl)cyclopentane-1,2-diol is represented by the formula (I) below.

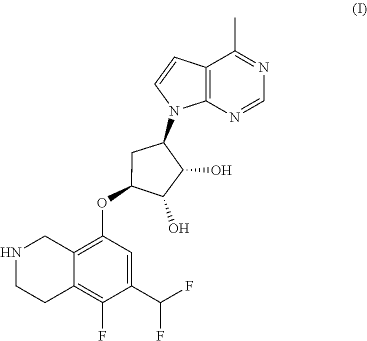 [SMILES: Cc1ncnc2c1ccn2[C@@H]1C[C@H](Oc2cc(C(F)F)c(F)c3c2CNCC3)[C@@H](O)[C@H]1O]

The preparation of the compound (1S,2S,3S,5R)-3-((6-(difluoromethyl)-5-fluoro-1,2,3,4-tetrahydroisoquinolin-8-yl)oxy)-5-(4-methyl-7H-pyrrolo[2,3- d]pyrimidin-7-y0cyclopentane-1,2-diol as a hydrochloride salt is described in Example 190 of WO2017/212385 and is depicted as follows.

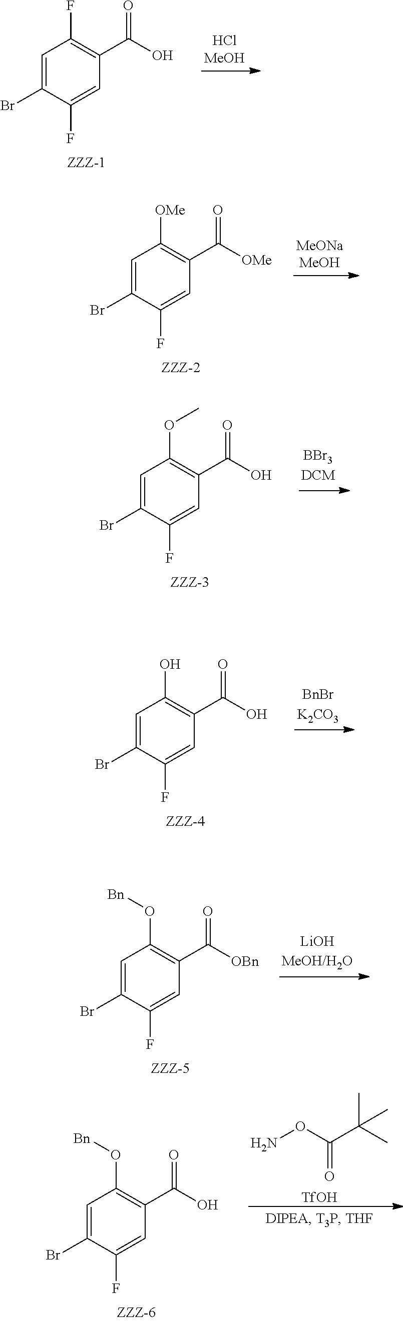

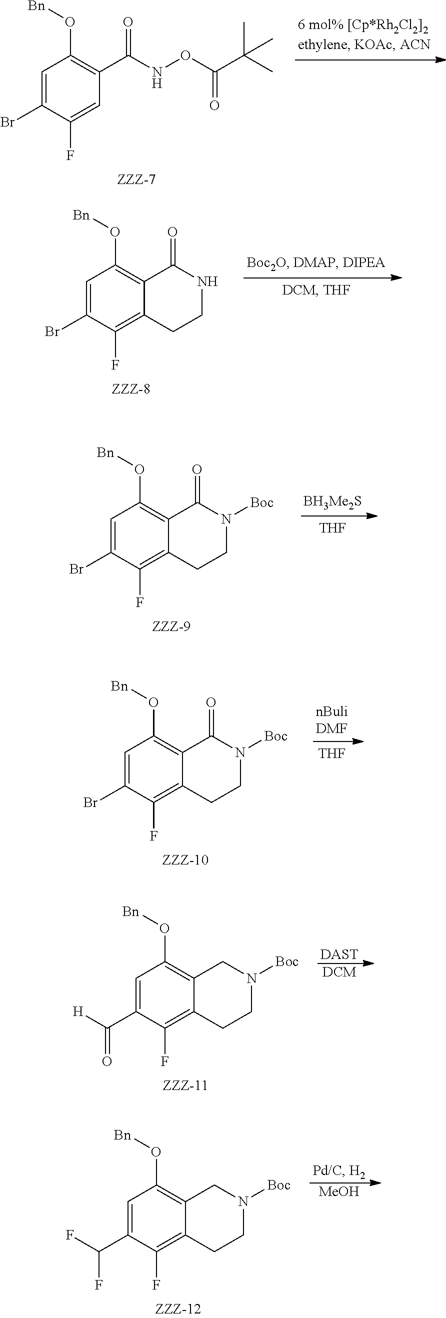

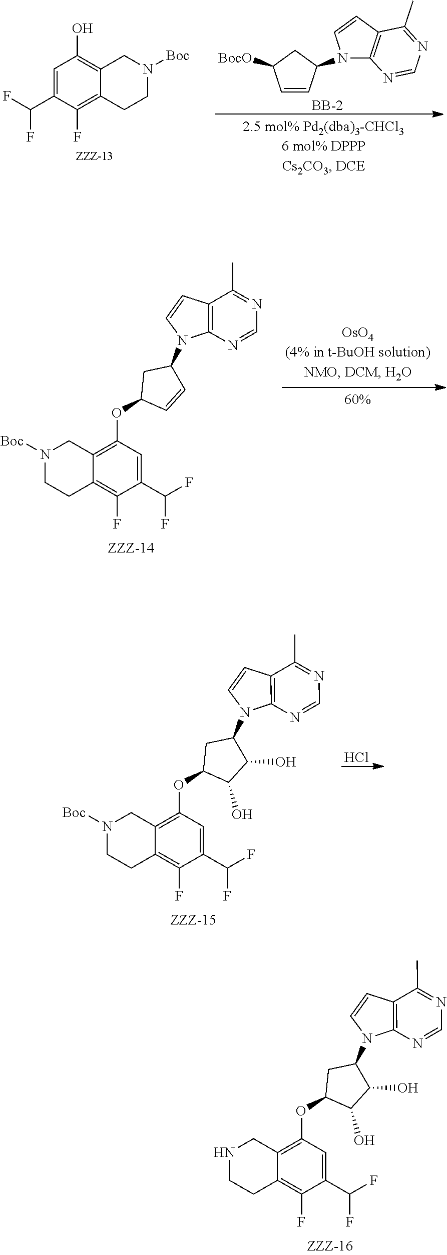

In this procedure tert-butyl 6-(difluoromethyl)-8-(((1S,2S,3S,4R)-2,3-dihydroxy-4-(4-methyl-7H-pyrrolo[2,3-d]pyrimidin-7-yl)cyclopentyl)oxy)-5- fluoro-3,4-dihydroisoquinoline-2(1H)-carboxylate is first deprotected in a mixture of dichloromethane and dioxane/HCl. The solid precipitated is separated, dried and lyophilized to provide a hydrochloride salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol as “a light yellow solid”. The specific solid form of the hydrochloride salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol prepared is not specified. However, the preparation of the compound (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol as a hydrochloride salt as described in Example 190 of WO2017/212385 was replicated as closely as possible in Reference Example 1 of PCT/IB2020/050397 (WO2020/152557), and PXRD and elemental analysis shows the product obtained in Example 190 of WO2017/212385 to be an amorphous, dihydrochloride having approximately 1 mol water per mol of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol. Certain other forms (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol may be prepared from this salt by standard basification techniques.

In WO2017/212385 (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol is described as a PRMT5 inhibitor useful in the treatment of abnormal cell growth in mammals, especially humans, particularly for the treatment of cancer. Human cancers comprise a diverse array of diseases that collectively are one of the leading causes of death in developed countries throughout the world (American Cancer Society, Cancer Facts and Figures 2005. Atlanta: American Cancer Society; 2005). The progression of cancers is caused by a complex series of multiple genetic and molecular events including gene mutations, chromosomal translocations, and karyotypic abnormalities (Hanahan & Weinberg, The hallmarks of cancer. Cell 2000; 100: 57-70). Although the underlying genetic causes of cancer are both diverse and complex, each cancer type has been observed to exhibit common traits and acquired capabilities that facilitate its progression. These acquired capabilities include dysregulated cell growth, sustained ability to recruit blood vessels (i.e., angiogenesis) and the ability of tumor cells to spread locally as well as metastasize to secondary organ sites (Hanahan & Weinberg 2000 above). Therefore, the ability to identify novel therapeutic agents that inhibit molecular targets that are altered during cancer progression, or target multiple processes that are common to cancer progression in a variety of tumors, presents a significant unmet need. Post-translational modification of arginine residues by methylation is important for many critical cellular processes including chromatin remodeling, gene transcription, protein translation, signal transduction, RNA splicing and cell proliferation. Arginine methylation is catalyzed by protein arginine methyltransferase (PRMT) enzymes. There are nine PRMT members in all, and eight have reported enzymatic activity on target substrates.

The protein arginine methyltransferase (PRMT) family of enzymes utilize S-adenosyl methionine (SAM) to transfer methyl groups to arginine residues on target proteins. Type I

PRMTs catalyze the formation of mono-methyl arginine and asymmetric di-methyl arginines, while Type II PRMTs catalyze mono-methyl arginine and symmetric di-methyl arginines. PRMT5 is a Type II enzyme, twice transferring a methyl group from SAM to the two ω-guanidino nitrogen atoms of arginine, leading to ω-NG, N'G di-symmetric methylation of protein substrates. PRMT5 protein is found in both the nucleus and cytoplasm, and has multiple protein substrates such as histones, transcription factors and spliceosome proteins. PRMT5 has a binding partner, Mep50 (methylosome protein 50) and functions in multiple protein complexes. PRMT5 is associated with chromatin remodeling complexes (SWI/SNF, NuRD) and epigenetically controls genes involved in development, cell proliferation, and differentiation, including tumor suppressors, through methylation of histones (Karkhanis, V. et al., Versatility of PRMT5 Induced Methylation in Growth Control and Development, Trends Biochem Sci 36(12) 633-641 (2011)). PRMT5 also controls gene expression through association with protein complexes that recruit PRMT5 to methylate several transcription factors-p53 (Jansson, M. et al., Arginine Methylation Regulates the p53 Response, Nat. Cell Biol. 10, 1431-1439 (2008)); E2F1 (Zheng, S. et al., Arginine Methylation-Dependent Reader-Writer Interplay Governs Growth Control by E2F-1, Mol Cell 52(1), 37-51 (2013)); HOXA9 (Bandyopadhyay, S. et al., HOXA9 Methylation by PRMT5 is Essential for Endothelial Cell Expression of Leukocyte Adhesion Molecules, Mol. Cell. Biol. 32(7):1202-1213 (2012)); and NFκB (Wei, H. et al., PRMT5 dimethylates R30 of the p65 Subunit to Activate NFκB, PNAS 110(33), 13516-13521 (2013)). In the cytoplasm, PRMT5 has a diverse set of substrates involved in other cellular functions including RNA splicing (Sm proteins), golgi assembly (gm130), ribosome biogenesis (RPS10), piRNA mediated gene silencing (Piwi proteins) and EGFR signaling (Karkhanis, 2011).

Additional papers relating to PRMT5 include: Aggarwal, P. et al., (2010) Nuclear Cyclin D1/CDK4 Kinase Regulates CUL4B Expression and Triggers Neoplastic Growth via Activation of the PRMT5 Methyltransferase, Cancer Cell 18: 329-340; Bao, X. et al., Overexpression of PRMT5 Promotes Tumor Cell Growth and is Associated with Poor Disease Prognosis in Epithelial Ovarian Cancer, J Histochem Cytochem 61: 206-217 (2013); Cho E. et al., Arginine Methylation Controls Growth Regulation by E2F1, EMBO J. 31(7) 1785-1797 (2012); Gu, Z. et al., Protein Arginine Methyltransferase 5 Functions in Opposite Ways in the Cytoplasm and Nucleus of Prostate Cancer Cells, PLoS One 7(8) e44033 (2012); Gu, Z. et al., Protein Arginine Methyltransferase 5 is Essential for Growth of Lung Cancer Cells, Biochem J. 446: 235-241 (2012); Kim, J. et al., Identification of Gastric Cancer Related Genes Using a cDNA Microarray Containing Novel Expressed Sequence Tags Expressed in Gastric Cancer Cells, Clin Cancer Res. 11(2) 473-482 (2005); Nicholas, C. et al., PRMT5 is Upregulated in Malignant and Metastatic Melanoma and Regulates Expression of MITF and p27(Kip1), PLoS One 8(9) e74710 (2012); Powers, M. et al., Protein Arginine Methyltransferase 5 Accelerates Tumor Growth by Arginine Methylation of the Tumor Suppressor Programmed Cell Death 4, Cancer Res. 71(16) 5579-5587 (2011); Wang, L. et al., Protein Arginine Methyltransferase 5 Suppresses the Transcription of the RB Family of Tumor Suppressors in Leukemia and Lymphoma Cells, Mol. Cell Biol. 28(20), 6262-6277 (2008).

PRMT5 is overexpressed in many cancers and has been observed in patient samples and cell lines including B-cell lymphoma and leukemia (Wang, 2008) and the following solid tumors: gastric (Kim 2005) esophageal (Aggarwal, 2010), breast (Powers, 2011), lung (Gu, 2012), prostate (Gu, 2012), melanoma (Nicholas 2012), colon (Cho, 2012) and ovarian (Bao, 2013). In many of these cancers, overexpression of PRMT5 correlated with poor prognosis. Aberrant arginine methylation of PRMT5 substrates has been linked to other indications in addition to cancer, such as metabolic disorders, inflammatory and autoimmune disease and hemaglobinopathies.

Given its role in regulating various biological processes, PRMT5 is an attractive target for modulation with small molecule inhibitors such as (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol.

### SUMMARY

Polymorphs are different crystalline forms of the same compound. The term polymorph may or may not include other crystalline solid state molecular forms including hydrates (e.g., bound water present in the crystalline structure) and solvates (e.g., bound solvents other than water present in the crystalline structure) of the same compound. Polymorphs typically have different crystal structures due to a different packing of the molecules in the lattice. This results in a different crystal symmetry and/or unit cell parameters which directly influences its physical properties such as the X-ray diffraction characteristics of crystals or powders.

Polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since any impurities present may produce undesired toxicological effects. Certain polymorphic forms may also exhibit enhanced (e.g. thermodynamic) stability or may be more readily manufactured in high purity in large quantities, and thus are more suitable for inclusion in pharmaceutical formulations. Certain polymorphs may display other advantageous physical properties such as lack of hygroscopic tendencies, improved solubility, and enhanced rates of dissolution due to different lattice energies. For pharmaceutical development and commercialization, there is a need to identify additional solid forms of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol, or a pharmaceutically acceptable salt or solvate thereof, that can be readily manufactured, processed and formulated. Consequently, there is a need to identify a solid form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol, or a pharmaceutically acceptable salt or solvate thereof, having desirable physicochemical and manufacturing properties. The present invention provides a novel crystalline form of a pharmaceutically acceptable salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol. More particularly, the present invention relates to a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol having desirable properties such as high crystallinity, high purity, and favorable physical stability, chemical stability, dissolution and mechanical properties. In particular, crystalline the mono-HCl salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol of the present invention provides improved physical stability (including low hygroscopicity) relative to the di hydrochloride salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol disclosed in WO2017/212385.

### BRIEF DESCRIPTION OF THE DRAWINGS

FIG. 1 shows the PXRD pattern of the “Form 4” crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol.

FIG. 2 shows the PXRD pattern of the “Form 3” crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol.

### DETAILED DESCRIPTION

The present invention may be understood more readily by reference to the following detailed description of the embodiments of the invention and the Examples and Figures included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

In one embodiment, the invention provides a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol.

In one embodiment, the invention provides a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol characterized by a PXRD pattern measured using Cu K-alpha (wavelength 1.54Å) radiation comprising characterizing peaks at about 11.0, 11.4 and 17.1 degrees 2-theta (+/−0.2 degrees 2-theta).

In one embodiment, the invention provides a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol characterized by a PXRD pattern measured using Cu K-alpha (wavelength 1.54Å) radiation comprising characterizing peaks at about 8.4, 11.0, 11.4 and 17.1 degrees 2-theta (+/−0.2 degrees 2-theta).

In one embodiment, the invention provides a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol characterized by a PXRD pattern measured using Cu K-alpha (wavelength 1.54Å) radiation comprising characterizing peaks at about 11.0, 11.4, 15.0 and 17.1 degrees 2-theta (+/−0.2 degrees 2-theta).

In one embodiment, the invention provides a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol characterized by a PXRD pattern measured using Cu K-alpha (wavelength 1.54Å) radiation comprising characterizing peaks at about 8.4, 11.0, 11.4, 15.0 and 17.1 degrees 2-theta (+/−0.2 degrees 2-theta).

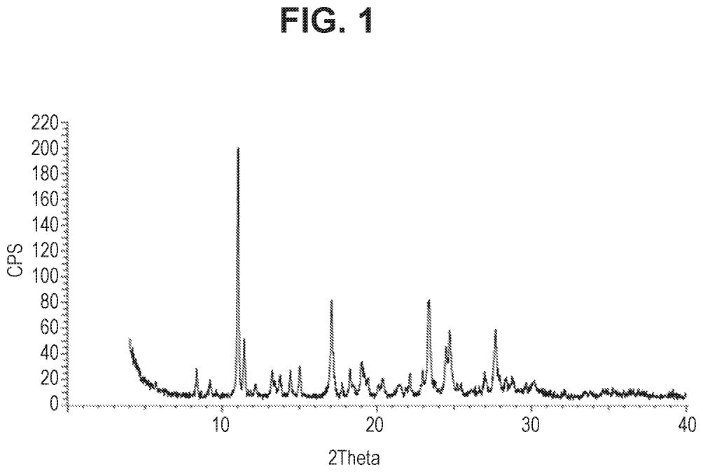

In another embodiment, the invention provides a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol characterized by a PXRD pattern measured using Cu K-alpha (wavelength1.54Å) radiation essentially the same as shown in FIG. 1.

In another embodiment, the invention provides a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol characterized by a PXRD pattern measured using Cu K-alpha (wavelength1.54Å) radiation having a PXRD peak listing essentially the same as in Table 1.

Each of the embodiments of the present invention described above can be combined with any other embodiment of the present invention described herein not inconsistent with the embodiment with which it is combined.

As used herein, the term:
“abnormal cell growth”, unless otherwise indicated, refers to cell growth that is independent of normal regulatory mechanisms (e.g., loss of contact inhibition). Abnormal cell growth may be benign (not cancerous), or malignant (cancerous). In frequent embodiments of the methods provided herein, the abnormal cell growth is cancer.
“cancer” refers to any malignant and/or invasive growth or tumor caused by abnormal cell growth. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of different type from the latter one.
“about” means having a value falling within an accepted standard of error of the mean, when considered by one of ordinary skill in the art.
“crystalline” means having three-dimensional order, i.e. a regularly repeating arrangement of molecules or external face planes. Crystalline forms (polymorphs) may differ with respect to thermodynamic stability, physical parameters, x-ray structure and characteristics, and preparation processes.
“essentially the same” means that variability typical for a particular method is taken into account. For example, with reference to X-ray diffraction peak positions, the term “essentially the same” means that typical variability in peak position and intensity are taken into account. One skilled in the art will appreciate that the peak positions (2θ) will show some variability, typically ±0.2°. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only. Similarly, Raman spectrum wavenumber (cm⁻¹) values show variability, typically as much as ±2 cm⁻¹, while ¹³C and ¹⁹F solid state NMR spectral peaks (ppm) show variability, typically ±0.2 ppm.
“mammal” refers to a human or animal subject. In certain preferred embodiments, the mammal is a human.
“hydrate”, in the context of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol means having a stoichiometric or non-stoichiometric amount of water bound in the crystal lattice by non-covalent intermolecular bonds. The hydrate state that has been observed to exist for this polymorph includes stoichiometry in the range of about 1.0 to about 1.4 molar equivalents of water per mole of the active moiety between 10% RH to 90% RH at 25° C.
“pharmaceutically acceptable” “carrier”, “diluent”, “vehicle”, or “excipient” refers to a material (or materials) that may be included with a particular active pharmaceutical agent to form a pharmaceutical composition, and may be solid or liquid. Exemplary of solid excipients or carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.
“substantially pure” is to be interpreted as the presence of equal or above 90%, equal or above 95%, equal or above 98%, or equal or above 99% weight/weight of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol as compared to any other physical form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol or a pharmaceutically acceptable salt or solvate thereof.
“therapeutically effective amount” refers to that amount of a compound being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, and/or (4) relieving to some extent (or, preferably, eliminating) one or more signs or symptoms associated with the cancer.
“treating”, as used herein, unless otherwise indicated, means reversing, alleviating, inhibiting the progress of, or preventing (i.e. prophylactic treatment) the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. With regard particularly to cancer, these terms simply mean that the life expectancy of an individual affected with a cancer will be increased or that one or more of the symptoms of the disease will be reduced.
term “2-theta value” or “2θ” refers to the peak position in degrees based on the experimental setup of the X-ray diffraction experiment and is a common abscissa unit in diffraction patterns. The experimental setup requires that if a reflection is diffracted when the incoming beam forms an angle theta (θ) with a certain lattice plane, the reflected beam is recorded at an angle 2-theta (2θ). It should be understood that reference herein to specific 2θ values for a specific polymorphic form is intended to mean the 2θ values (in degrees) as measured using the X-ray diffraction experimental conditions as described herein. For example, as described herein, Cu K-alpha 1 (wavelength 1.54Å) was used as the source of radiation.

Also provided by the present invention is a pharmaceutical composition comprising a crystalline form as described herein and a pharmaceutically acceptable carrier or excipient.

Additionally, provided by the present invention are methods of treatment of abnormal cell growth in a mammal comprising administering to the mammal a therapeutically effective amount of a crystalline form as described herein, or composition thereof.

Further provided by the present invention is a crystalline form as described herein, or composition thereof, for use as a medicament or for use in the treatment of abnormal cell growth in a mammal.

Further still, the present invention provides for the use of a crystalline form as described herein, or composition thereof, for the preparation of a medicament useful in the treatment of abnormal cell growth in a mammal.

The abnormal cell growth may be cancer. The cancer referred to herein may be lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma.

The crystalline form as described herein can be administered alone or as a formulation in association with one or more pharmaceutically acceptable carriers or excipients. The choice of excipient will to a large extent depend on factors such as the particular mode of administration, the effect of the excipient on solubility and stability, and the nature of the dosage form.

It will be appreciated that when a crystalline form as described herein is dissolved for formulation purposes, the crystal lattice is no longer present. In this situation the reference to active compound of a crystalline form as described herein below means the (therapeutically active) compound of a crystalline form as described herein.

Pharmaceutical compositions suitable for the delivery of the crystalline form as described herein and their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The crystalline form as described herein may be administered orally. Oral administration may involve swallowing, so that the crystalline form enters the gastrointestinal tract, or buccal or sublingual administration may be employed by which the crystalline form enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The crystalline form as described herein may also be used in fast-dissolving, fast-disintegrating dosage forms such as those described in Expert Opinion in Therapeutic Patents, 11 (6), 981-986 by Liang and Chen (2001). For tablet dosage forms, depending on dose, the crystalline form as described herein may make up from 0.5 wt (weight) % to 80 wt % of the dosage form, more typically from 0.5 wt % to 20 wt % of the dosage form. In addition to the drug, the tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 2 wt % to 10 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets contain up to about 80 wt % of a crystalline form as described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled targeted- and programmed-release.

Suitable modified release formulations are described in U.S. Pat. No. 6,106,864. Details of other suitable release technologies such as high energy dispersions and osmotic and coated particles can be found in Verma et al, Pharmaceutical Technology On-line, 25(2), 1-14 (2001). The use of chewing gum to achieve controlled release is described in WO 00/35298.

The crystalline form as described herein may also be administered directly into the blood stream, into muscle, or into an internal organ. Suitable means for parenteral administration include intravenous, intra-arterial, intraperitoneal, intrathecal, intraventricular, intraurethral, intrasternal, intracranial, intramuscular and subcutaneous. Suitable devices for parenteral administration include needle (including micro-needle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of the crystalline form as described herein used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted- and programmed-release. Thus a crystalline form as described herein may be formulated as a solid, semi-solid, or thixotropic liquid for administration as an implanted depot providing modified release of the active compound. Examples of such formulations include drug-coated stents and PGLA microspheres.

The crystalline form as described herein may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and micro-needle or needle-free (e.g. Powderject™, Bioject™, etc.) injection.

Formulations for topical administration may be formulated to be immediate and/or modified release. Modified release formulations include delayed-, sustained-, pulsed-, controlled targeted- and programmed-release. The crystalline form as described herein can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may include a bioadhesive agent, for example, chitosan or cyclodextrin.

The pressurized container, pump, spray, atomizer, or nebulizer contains a solution or suspension of the crystalline form as described herein comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the crystalline form as described herein, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the crystalline form as described herein per actuation and the actuation volume may vary from 1 μL to 100 μL. A typical formulation includes a crystalline form as described herein, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid) (PGLA). Modified release formulations include delayed-, sustained-, pulsed-, controlled-, targeted- and programmed-release.

In the case of dry powder inhalers and aerosols, the dosage unit is determined by means of a valve which delivers a metered amount. Units in accordance with the invention are typically arranged to administer a metered dose or “puff” containing a desired mount of the crystalline form as described herein. The overall daily dose may be administered in a single dose or, more usually, as divided doses throughout the day.

A crystalline form as described herein may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted- and programmed-release.

A crystalline form as described herein may also be administered directly to the eye or ear, typically in the form of drops of a micronized suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted-, or programmed-release.

A crystalline form as described herein may be combined with soluble macromolecular entities, such as cyclodextrin and suitable derivatives thereof or polyethylene glycol-containing polymers, in order to improve solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in WO 91/11172, WO 94/02518 and WO 98/55148.

The amount of the active compound of a crystalline form as described herein to be administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day.

Inasmuch as it may desirable to administer a combination of a crystalline form as described herein and a further anti-cancer compound, for example, for the purpose of treating a particular disease or condition, it is within the scope of the present invention that two or more pharmaceutical compositions, at least one of which contains a active compound of a crystalline form as described herein, may conveniently be combined in the form of a kit suitable for co-administration of the compositions. Thus the kit of the invention includes two or more separate pharmaceutical compositions, at least one of which contains a crystalline form as described herein, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid. The present invention is described with reference to the following Examples. It is to be understood that the scope of the present invention is not limited by the scope of the following Examples.

### Example 1

Synthesis of Crystalline Mono-HCl Salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol (Form 3)

Step 1: In a 5-dram vial was added (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol (100 mg, 0.223 mmol) and then methanol was added dropwise. The mixture turned into a yellow solution. The mixture precipitated a white solid. Continuing stirring at RT then heated to 70° C. for 15 minutes and then cooled to RT and stirred at 3 hours. Solid were filtered and washed with methanol allowing lot 00110639-3370-001 (74 mg, solids 68%) as white solids.

Step 2: To a solution of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol (2000.0 mg, 3.588 mmol) (disclosed in WO 2017/212385A1, ZZZ-16) in distilled water (7.62 mL, c=0.2 M) was added NaHCO₃ (20.0 mL, c=0.090 M). 10% MeOH/EtOAc was extracted with 5×50 mL until aqueous did not contain product. Dry organics were filtered and concentrated over Na₂SO₄ and then lyophilized to obtain a white solid using MeOH and water. (1582.0 mg, 98.3%).

Step 3: To a solution of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol (850 mg, 1.52 mmol) (disclosed in WO 2017/212385 Al, ZZZ-16) in distilled water (7.62 mL, c=0.2 M) was added NaHCO₃ (20 mL, c=0.038 M). 10% MeOH/EtOAc was extracted 4×40 mL. Dry organics were filtered and concentrated over Na₂SO₄ and then lyophilized to obtain a white solid (625.0 mg, 91.4%).

Step 4: To a solution of the product of Step 3 (100 mg, 0.223 mmol) was added ˜3 mg of seed from the product of Step 1 and stirred at ˜200 rpm. HCl (2M in EtOH) was added and stir at RT at ˜200 rpm. Mixture was stirred at this speed for >48 hours. The obtained solids (with 0.64 eq EtOH) were filtered and washed with 2 mL EtOH.

Step 5: The product of Step 2 (1.6 g) and the product of Step 3 (0.5 g) were combined in ethanol (47.4 ml, 0.1M) (at first, was 0.2M) in a 150 mL pressure vessel. Material was not fully soluble. The system was sonicated, and material precipitated as a white solid. The remaining 24 mL were added but system was still a suspension even upon heating. ˜5mg seeds of the Step 4 product were added. The reaction was stirred at ˜200 rpm. HCl (2M in EtOH) (164 mg, 4.5 mmol, 2.25 ml, 2.0M) was added. The materials appeared to be ˜90% in solution, and then white solids precipitated. The system was heated to 60° C. for 1 hour then cooled back down and stir 200 rpm overnight. An aliquot was taken and diluted with 5 mL MTBE, then filtered. The reaction vessel was diluted with 35 mL of TBME and stirred for 15 min. A white solid was obtained and filtered.

Step 6: To a solution of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol (1000 mg, 2.05 mmol) in EtOH (22 mL) was added seed from the product of Step 5 (5 mg). 2 N HCl in EtOH (1.03 mL) was added dropwise at 15° C. The mixture was stirred at 15° C. for 30 min then stirred at 60° C. for 30 min. The mixture was then stirred at 10° C. for 16 h. The mixture was diluted with TBME (5 mL) and stirred at 15° C. for 15 min. The mixture was filtered and the obtained solid was washed with TBME and dried in vacuo at 60° C. for 2 h to afford a white solid (800 mg, 80.4%).

Step 7: To a solution of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol (86 g, 180 mmol) in EtOH (1892 mL) stirred with mechanical stirrer (200 r/min) was added the product of Step 6 (400 mg). 2 N HCl in EtOH (90.1 mL) was added dropwise at 15° C. The mixture was stirred at 15° C. for 30 min then stirred at 60° C. for 30 min then at 15° C. for 30 min and at 60° C. for 15 min. The mixture was stirred at 15° C. for 16 h. The mixture was diluted with TBME (1500 mL) and stirred at rt 15° C. for 15 min. The mixture was filtered and the solid was washed with TBME and dried in vacuo at 60° C. for 2 h to afford a white solid (82 g, 93.8%) then concentrated in vacuo.

Step 8: The product of Step 7 (1.620 g) was added to 5 mL of Mili-Q Water in a 10 mL round bottom flask. The solution was stirred for a few minutes until it solidified. 2 mL of Mili-Q Water was added, vortexed, and then the slurried mixture was stirred at room temperature for 5 days before isolating a solid which was filtered using vacuum filtration and a Buchner funnel, and washed sample with approx. 2.0 mL of Mili-Q water in order to collect all white solid from round bottom flask on a filter paper. The solid was placed under vacuum at room temperature to dry overnight.

### Example 2

PXRD analysis of Form 3 Crystalline Mono-HCl (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol

The product of Example 1, Step 8 was analyzed by elemental analysis and shown to be consistent with mono hydrochloride and 2.4 equivalents of water. The sample was also analyzed by PXRD. Powder X-ray diffraction analysis was conducted using a Bruker A25 D8 Advance diffractometer equipped with a Cu radiation source. Diffracted radiation was detected by a Lynxeye detector. The X-ray tube voltage and amperage were set at 40 kV and 40 mA respectively. Data was collected in the Theta-2-Theta goniometer in a locked couple scan at the Cu wavelength from 4.0 to 40.0 degrees 2-Theta using a step size of 0.020 degrees and a step time of 0.100 second. Samples were prepared by adding sample into a silicon low background sample holder and flattening with spatula. Data analysis was performed by EVA diffract plus software. The peak selection carried out manually was checked to ensure that all peaks below 30° 2θ had been captured and all peak positions had been accurately assigned. A typical error of ±0.2° 2θ in peak positions (USP-941) applies to this data. The minor error associated with this measurement can occur because of a variety of factors including: (a) sample preparation (e.g., sample height), (b) instrument characteristics, (c) instrument calibration, (d) operator input (e.g. in determining the peak locations), and (e) the nature of the material (e.g. preferred orientation and transparency effects).

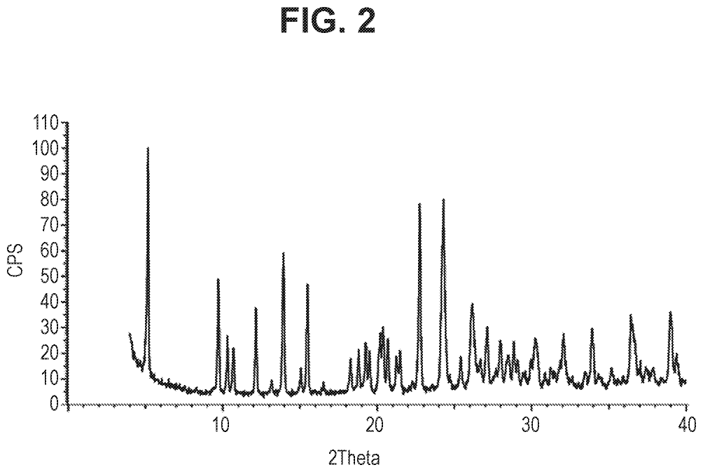

The PXRD profile for the crystalline form of the HCl salt Form 3 is provided in FIG. 2 and the peak list in Table 1.

TABLE 1

| Angle | Rel. Intensity, % |
|---|---|
| 5.2 | 100 |
| 9.7 | 50 |
| 10.3 | 25 |
| 10.7 | 21 |
| 12.2 | 38 |
| 13.2 | 7 |
| 13.9 | 56 |
| 15.1 | 11 |
| 15.5 | 48 |
| 18.3 | 14 |
| 18.8 | 19 |
| 19.3 | 21 |
| 19.5 | 17 |
| 20.2 | 22 |
| 20.4 | 28 |
| 20.7 | 26 |
| 21.2 | 16 |
| 21.5 | 19 |
| 22.7 | 86 |
| 24.3 | 84 |
| 25.4 | 12 |
| 26.2 | 34 |
| 27.1 | 28 |
| 28.0 | 22 |
| 28.5 | 11 |
| 28.8 | 17 |
| 29.1 | 9 |

### Example 3

Synthesis of Crystalline Mono-HCl Salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol (Form 4)

Approximately 156.0 mg of Form 3 (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol were added to a 20 mL scintillation vial with a stir-bar and 5 mL of 1-butanol and stirred at room temperature. The solution was allowed to slurry for 3 days before isolating solid (checking aliquots by PXRD to confirm complete conversion). Solids were isolated using vacuum filtration on a filter paper in a Buchner funnel. The sample was washed with approximately 2 mL of 1-butanol and then left sample to air dry. Then the sample was dried at 80° C. in the oven for 3 days and analyzed by elemental analysis and PXRD. The elemental analysis data concluded that sample is consistent with mono hydrochloride and 0.88 eq of water.

### Example 4

Pharmaceutical formulations of Form 3 and Form 4 Crystalline Mono-HCl Salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol

Prototype formulation blends comprising Form 3 or Form 4 crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol may be prepared using conventional excipients commonly used in pharmaceutical tablet formulations. Tablets typically contain from 0.5-30% wt/wt of crystalline (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol. Microcrystalline cellulose and dibasic calcium phosphate anhydrous may be used as tablet fillers, and sodium starch glycolate may be used as a disintegrant. Magnesium stearate may be used as a lubricant. A typical tablet formulation is provided in Table 2.

TABLE 2

| Component | Role | wt/wt % |
|---|---|---|
| Form 4 Crystalline Mono-HCl (1S,2S,3S,5R)-3- | API | 3.8 |
| ((6-(difluoromethyl)-5-fluoro-1,2,3,4- | | |
| tetrahydroisoquinolin-8-yl)oxy)-5-(4-methyl-7H- | | |
| pyrrolo[2,3-d]pyrimidin-7-yl)cyclopentane-1,2- | | |
| diol | | |
| Cellulose | Filler | 61.4 |
| [Avicel PH 102 (Trade Mark)] | | |
| Dibasic Calcium Phosphate | Filler | 30.8 |
| [DiCAFOS A12 (Trade Mark)] | | |
| Sodium starch glycolate | Disintegrant | 3.0 |
| [Explotab (Trade Mark)] | | |
| Magnesium stearate | Lubricant | 1.0 |

### Example 5

PXRD analysis of Form 4 Crystalline Mono-HCl (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol

A Powder X-ray diffraction analysis was conducted using a Bruker A25 D8 Advance diffractometer equipped with a Cu radiation source. Diffracted radiation was detected by a Lynxeye detector. The X-ray tube voltage and amperage were set at 40 kV and 40 mA respectively. Data was collected in the Theta-2Theta goniometer in a locked couple scan at the Cu wavelength from 4.0 to 40.0 degrees 2-Theta using a step size of 0.020 degrees and a step time of 0.100 second. Samples were prepared by adding sample into a silicon low background sample holder and flattening with spatula. Data analysis was performed by EVA diffract plus software. The peak selection carried out manually was carefully checked to ensure that all peaks below 30° 2θ had been captured and all peak positions had been accurately assigned. A typical error of ±0.2° 2θ in peak positions (USP-941) applies to this data. The minor error associated with this measurement can occur because of a variety of factors including: (a) sample preparation (e.g., sample height), (b) instrument characteristics, (c) instrument calibration, (d) operator input (e.g. in determining the peak locations), and (e) the nature of the material (e.g. preferred orientation and transparency effects). The PXRD profile for the crystalline form of the HCl salt Form 4 is provided in FIG. 1 and the peak list in Table 3.

TABLE 3

| Angle | Rel. Intensity, % |
|---|---|
| 8.4 | 11 |
| 9.2 | 6 |
| 11.0 | 100 |
| 11.4 | 25 |
| 12.1 | 5 |
| 13.2 | 12 |
| 13.8 | 8 |
| 14.4 | 9 |
| 15.0 | 11 |
| 17.1 | 42 |
| 17.8 | 8 |
| 18.3 | 11 |
| 19.0 | 12 |
| 20.4 | 6 |
| 21.4 | 4 |
| 22.1 | 8 |
| 23.0 | 9 |
| 23.4 | 31 |
| 24.5 | 17 |
| 24.7 | 25 |
| 27.0 | 7 |
| 27.7 | 26 |

### * * *

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention.

1. A crystalline mono-HCl salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol.

2. The A crystalline mono-HCl salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol of claim 1 characterized by a PXRD pattern measured using Cu K-alpha radiation comprising one or more peaks at about 11.0, 11.4 and 17.1 degrees 2-theta (+/−0.2 degrees 2-theta).

3. The A crystalline mono-HCl salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol of claim 1 characterized by a PXRD pattern measured using Cu K-alpha radiation comprising one or more peaks at about 8.4, 11.0, 11.4 and 17.1 degrees 2-theta (+/−0.2 degrees 2-theta).

4. The A crystalline mono-HCl salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol of claim 1 characterized by a PXRD pattern measured using Cu K-alpha radiation comprising one or more peaks at about 11.0, 11.4, 15.0 and 17.1 degrees 2-theta (+/−0.2 degrees 2-theta).

5. The A crystalline mono-HCl salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol of claim 1 characterized by a PXRD pattern measured using Cu K-alpha radiation comprising peaks at about 8.4, 11.0, 11.4, 15.0 and 17.1 degrees 2-theta (+/−0.2 degrees 2-theta).

6. (canceled)

7. The A pharmaceutical composition comprising a crystalline mono-HCl salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol as claimed in claim 1 and a pharmaceutically acceptable carrier or excipient.

8. A crystalline mono-HCl salt of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol as claimed in claim 1, or a composition thereof as claimed in claim 7, for use as a medicament.

9. A crystalline mono HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol as claimed in claim 1, or a composition thereof as claimed in claim 7, for use in the treatment of abnormal cell growth in a mammal.

10. (canceled)

11. A method of treatment of abnormal cell growth in a mammal comprising administering to the mammal a therapeutically effective amount of a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol as claimed in claim 1.

12. (canceled)

13. A combination of a crystalline mono-HCl salt form of (1S,2S,3S,5R)-3-((6-(difluoromethyl)-5-fluoro-1,2,3,4-tetrahydroisoquinolin-8-yl)oxy)-5-(4-methyl-7H-pyrrolo[2,3-d]pyrimidin-7- yl)cyclopentane-1,2-diol as claimed in claim 1, with another anti-cancer agent.
